(19) United States
(12) Patent Application Publication
(10) Pub. No.: US 2021/0325415 A1
(43) Pub. Date: Oct. 21, 2021

(54) Infectious Microbe Detection Method and Apparatus

(71) Applicant: Liberty Health Technologies, Inc., Santa Barbara, CA (US)

(72) Inventor: Chris Outwater, Carpinteria, CA (US)

(21) Appl. No.: 17/234,548

(22) Filed: Apr. 19, 2021

Related U.S. Application Data
(60) Provisional application No. 63/012,499, filed on Apr. 20, 2020.

(51) Int. Cl. G01N 35/00; G05B 13/02; C12Q 1/70; C12Q 1/37

(52) U.S. Cl. CPC G01N 35/00871; G05B 13/0265; G05D 1/021; C12Q 1/37; C12Q 1/70

(57) ABSTRACT

The invention is a novel and economical method for detecting infectious microbes, whether airborne or bound to surfaces. A microbe is any microorganism, especially a virus, or bacteria, or fungal spore of any kind. Every infectious microbe has a specific path or vector to invade the human body and cause disease. This inspection and detection method utilizes a real or simulated human cell enzyme coating to bind the target microbe to the sample gathering filter or swab. This greatly increases the sample gathering sensitivity to the target microbe.

### CROSS REFERENCE TO RELATED APPLICATIONS

This application claims the benefit of U.S. Provisional Application Ser. No. 63/012,499, filed Apr. 20, 2020, which is hereby incorporated by reference in its entirety.

### BACKGROUND OF THE INVENTION

The invention is a novel and economical method for detecting infectious microbes, whether airborne or bound to surfaces. A microbe is any microorganism, especially a virus, or bacteria, or fungal spore of any kind. Every infectious microbe has a specific path or vector to invade the human body and cause disease. This inspection and detection method utilizes a real or simulated human cell enzyme coating to bind the target microbe to the sample gathering filter or swab. This greatly increases the sample gathering sensitivity to the target microbe.

At the current stage in the detection technology, the actual processing and microbe detection and reporting requires between 20 and 40 minutes and takes place at a site remote from the sample gathering site. However, on-site processing and detection will be available in the near future, allowing this microbe sampling and detection to be reported in virtually real time.

### SUMMARY OF THE INVENTION

This invention describes a novel method for gathering samples both from air and direct contact with surfaces at a specified time and place in order to rapidly process said samples and determine with an acceptable amount of certainty that an infectious microbe is present in the specified space at that time and to adjust human traffic and occupancy accordingly.

### DETAILED DESCRIPTION OF THE INVENTION

### Two Microbe Detection Modalities

The first is a specially designed bio-active, air capture filter and its secure, airtight tray compartment designed within an aerial drone (VDrone) or a land-based robot. The second is a specially designed bio-active, contact capture swab and its secure, airtight tray compartment designed within an aerial drone or a land-based robot. Therefore, this disclosure describes both modalities, air samples and direct contact swabs, as contained and carried in a specially designed aerial drone or a ground-based mobile robot device. The bio-active filter and secure compartment is further described below.

### Programming and Operations and Control of the Ground-Based Robot or VDrone

In the ground-based embodiment, the vehicle could be a land-based robot such as a modified robot vacuum cleaner or a surveillance robot. The robot can be remote controlled, or have a pre-programmed search area or could also be autonomous and running continuously even in a high traffic area as long as it has warning lights at a proper height, such as 6 feet, and proper sounds so that a human would not purposefully walk into it or bump it. Even if this accidental contact were to happen, it is very likely that neither the human nor the robot would be harmed and both could continue on their desired paths. The VDrone could be operated in a similar manner, but kept well above human height so that there would be almost no chance of accidental human contact. When it is safe to do so during low or no human occupancy, the VDrone could venture down to capture air samples closer to human head level or to drag a swab along an object such as a bench or seat, etc.

### The Specially Designed Recharging Station for Batteries, Filters and Swabs

The land-based robot could gather samples in two modes; similar to the aerial drone. One mode is to whisk air towards a filter that has negative pressure on the backside so that the air and any particles, including infectious microbes, are directed at and through the bio-active filter. It is not required that the sample air pass through the filter. As long as, like with the VDrone, the sample air is directed at the surface of the bio-active air filter. The filter can be approximately 8 cm in diameter, or 8 cm square; however, the exact size of the filter can vary and is not crucial to the effective operations of gathering microbe samples.

In one embodiment, the whisks are not actually sampled, only the bio-active air filter is sampled. The whisks could be removed and stored or disposed of after each designated “search route” is terminated. In this embodiment, although the whisks must be removed following each route so the used whisks do not contaminate the next route, the whisks themselves are not bio-active and are not directly processed for PCR and CRISPR processing. In a preferred embodiment, and similar to the VDrone-transported swab, the land-based robot's whisk can be a bio-active swab or micro-mop and while recharging at the battery recharging station the VDrone's or robot's bio-active swab or swabs (depending on how many search routes were run) are ejected from the VDrone's or robot's secure, airtight transport tray compartment and deposited into a similar secure, airtight tray compartment in the recharging station. In another embodiment, the tray compartment itself transfers from the VDrone or land-based robot to the recharge station and a new tray compartment is added to the VDrone or robot. In all cases, the sample is maintained in a controlled environment, such that temperature, humidity, amount of ambient light, etc., is maintained to preserve the life of the target microbes.

When appropriate, such as following delivery of sample tray compartments, a signal is automatically generated at the recharge station, which has communication with a remote server and control center, requesting pickup of samples and delivery to a secure, remote processing center. At the processing center, as soon as the target microbe presence is detected, the results are sent automatically to the designated health and safety administrators via multiple channels, such as voice message, text message, or email. A special alert will be assigned to the message if the designated search microbe is detected. Threshold amounts, if desired, can be tested and determined over time.

In another embodiment, the station, or a connected processing system can locally perform PCR and a sample detection process using a system based on CRISPR technology. The local station would then communicate to the administrators as described in the above paragraph. One company offering such a CRISPR system is Mammoth Biosciences in California. See https://mammoth.bio/2020/02/15/white-paper-a-protocol-for-rapid-detection-of-sars-cov-2-using-crispr-sars-cov-2-detectr/.

In both modalities, at the end of their search route the VDrone or ground-based robot returns to a specially designed recharge and filter or swab deposit station where the drone or robot ejects the designated filter(s) or swab(s) or the tray compartment(s) into an environmentally safe and secure storage device.

Also, the VDrone or ground-based robot is replenished with new, unexposed, bio-active swabs and filters, as needed. New (unexposed) and used (exposed) bio-active swabs and filters will have a definite shelf life, even if held in microbe-preferential controlled environments. Recharge stations are visited periodically either by a human, a robot or a drone in order to replenish said time-sensitive filters and swabs.

As mentioned, in later versions of this invention, the actual PCR and CRISPR processing can take place locally within the station or in a device securely connected to the recharging and replenishing station.

Detection reports can be sent urgently and securely via wired or wireless communications to the authorized health and safety administrator for that district or building.

The VDrones have search zones and routes and are fitted with GPS or local area, radio frequency-based wayfinding technology within a defined space, either internal/indoor or external/outdoor likely at a high density human occupancy area, such as a lobby, or transportation station, an airport, a foyer, or inside a mass transit vehicle such as a train, plane, or bus, etc.

In one embodiment, the VDrone maps and learns a target space where humans are, or will be, congregating. Using machine learning and air direction detectors based on heating, ventilation, and air conditioning (HVAC) measurements, VDrones and robots can be focused on certain areas and spaces at key times of the day, perhaps prior to planned massive amounts of human traffic and occupancy, such as a major event or at a transportation hub. This aspect of both machine learning and drone and robot space mapping is understood by anyone skilled in the art of the drone and robot industry. Drones and robots can be controlled remotely or can be programmed to map a specific space for multiple return routes. Both modalities could be used to gather samples of infectious microbes.

Note that a smoke or inert particle spray, perhaps luminescent, can be used for a pre-installation air current study and can be part of the overall planned detection system. The luminescent particles or smoke could be delivered from a drone, for example, and as the air settles, general air current patterns caused by HVAC could be observed and this information used to direct routes for sampling.

Designated sampling locations are tracked in a database so that they can be traced to their correlated air or contact swab samples. A large inspection space can be divided into unique inspection sublocations.

In one embodiment, without compromising airworthiness, a special undercarriage structure is added to the drone to force the downward flowing air from each rotor into a centrally located common air sample capture filter that sits in its compartment below the main chassis. This is where a camera or other payload would normally be located. This does not mean that there may not be a camera or other sensor(s) on the same drone at the same time. In another embodiment filter compartments could be located below each rotor. Thus the device can gather air samples and accumulate any microbes on the specially designed filter in a specified space over a predetermined period of time.

### Special Bio-Active Filter and Swab

An important aspect of the VDrone is that the special filter is coated and imbued with a biosimilar human cell culture that matches the target microbe's entry vector within the human body. Angiotensin-converting enzyme 2 (ACE2) is a zinc-containing metalloenzyme surrounding the ACE2 cell membrane. The cell membranes supporting these enzymes are the target vector for the SARS-based COVID 19 disease. There are two embodiments for the special biosimilar enzyme coatings; in one embodiment the ACE2 cells will be viable in the coating for a predetermined period of time after coating the filter. In another embodiment, only the ACE2 surrounding enzyme is present in the special filter bio-coating and the ACE2 cells do not need to be present and/or viable. In this embodiment, the use of the specially coated filters could be less expensive and also less time sensitive. The special enzyme filter and swab coating process can use a dip, a spray technique, or any other feasible manner to coat the bio-active and biosimilar coating onto the said filter or swab.

It is known that specific genes will bind with the COVID virus. These are the N-gene (SARS-CoV-2 specific)•E-gene (SARS-CoV, bat-SARS-like-CoV, and SARS-CoV-2 coronaviruses). In one embodiment, at least one of these genes, or a plurality of genes, will be included in the bio-active coating on the filter or swab.

A company such as Creative Biogene of Shirley, N.Y. could provide the specific target cells and/or the target enzymes that will bind the infectious microbe to the bio-active filter and swab. See https://www.creative-biogene.com/products/coronavirus-receptor-stable-cell-lines.html?gclid=Cj0KCQjwyur0BRDcARIsAEt86IASMJBPIDCRM_6dSB92RsTjVxRv0POw1D4DBU4g2JG0SNMslQ_Sm18aAuikEALw_wcB.

This microbe binding bio-active coating will be varied as needed and can be designed for specific infectious microbe vectors as they emerge into the human population. In this embodiment, the human biosimilar ACE 2 cell outer coating, or in another embodiment the cells themselves are added to a mucous-like vehicle and are coated onto a stable carrier in the form of a filter or swab. It is also feasible that a dry carrier will be developed to accept the bio-active coating. The air sample capture device filter in the VDrone or in the land-based robot will be exposed to large quantities of pressurized air directed to the active surface of the filter.

In some cases, the amount of air will need to be controlled. We will need to have enough airflow to capture the maximum number of microbes entering the airflow, but not so much airflow that it hinders the target microbe, in this instance the SARS coronavirus leading to COVID 19 disease, from binding to the surface of the filter. Air flow can be measured for calculation of parts per million or billion in the target space or area. In the near term, we only need to know if the infectious microbe is detectable and therefore deemed present in the air or on surfaces in those detectable amounts and in an overabundance of caution the authorities will assume that even the smallest detectable amount of an infectious microbe could lead to human infections. Certainly, as we learn more about specific microbes, reasonable thresholds can be determined for designated traffic and human aggregation areas and times.

In one embodiment, the filter structure that holds the bio-active coating can be made of a reusable inert base material such as stainless mesh. In a preferred embodiment, the filter structure could be composed of a disposable cloth or paper type material. In another embodiment, a filter or swab base material is composed of a substance that can be dissolved away in solution without affecting the target infectious microbe that is bound to the coating.

In another embodiment, the air filter could be stationary and mounted to a wall or ceiling or window and the bio-active filter could be removed periodically for inspection from the stationary device holding the filter. The filter can be transferred in a bio-secure container and sent for remote processing and detection purposes.

In a preferred embodiment there is an air filter and in addition there is also a mechanism for a bio-active swab. The bio-active swab will be in direct contact with the target surfaces and after a predetermined period of time on its search route, the VDrone or robot returns to the recharge device and deposits the exposed filters and swabs and also receives new filters and swabs prior to the next designated search route.

In another embodiment, there is negative air pressure on the back surface of the air filter. This could be in the VDrone or in the land-based robot, for example. This is to draw large quantities of sample air from a specified location, such as a lobby or mass transit vehicle interior space at a specified time, such as before the space is inhabited with a large number of humans. The sample air can be forced and directed at the air capture surface and/or pulled through the bio-active capture filter by a suction device.

The VDrone's filter compartment can be almost any shape, but will likely be either square or round due to the air pattern of the overhead rotors. In other words, the downward thrust airflow from the rotors will be for the most part equal, whether there are four or six rotors. The compartment's air flow door can be opened and closed remotely in real time, or this function can be programmable, based on the designated space that the VDrone is inspecting and sampling.

Whether a drone or land-based robot, it is important that the system knows exactly which space or area has been sampled; no more and no less, and at what time. Therefore, the filter compartment or the swab needs to be securely compartmentalized in an airtight, sealed compartment so the sample space and time can be determined.

In another embodiment, we do not use the airflow from the rotors and the VDrone filter compartment has its own fan to direct air onto the filter surface and/or a suction device to pull the sample air through the filter.

In another embodiment, the VDrone will be pre-programmed to fly around and sample a specific, well mapped space, such as a large foyer of a bank or other large office building that is highly trafficked by humans at known hours. When it is safe to do so, the VDrone will periodically descend to the average human head height to sample the air at that specific level.

In a preferred embodiment, the drone can also lower itself and drag a disposable specimen capture swab along a surface such as a table, chair, couch, handrail, seat, etc. The swab could be on a flexible extender that would be better suited to follow the contours of an uneven surface and allow the VDrone to maintain a more or less even altitude as it travels above the surface of an object being swabbed. The swab can be composed of filaments like a micro mop, approximately 3 centimeters square in size. The size of the swab can vary, based on the size and lifting power of the specific VDrone and the specified sampling space. The exact size is not critical to the intended sampling action of the swab as the VDrone drags it along a target surface. As mentioned, the capture swab or mop can have various shapes. In the preferred embodiment, the swab is in the shape of a small mop with multiple filaments or tentacles thus offering a greater overall sample gathering surface. The swab can be controlled in real time by remote control, or be programmed to drop and gather samples, based on the designated space and time. Upon terminating its search route, the swab will be drawn back inside the VDrone's secure, air tight, compartment before it returns to the recharging and sample drop off and sample replenishing station.

At this recharge and replenishment stage, in addition to recharging its batteries, the VDrone or land-based robot exchanges exposed samples for new, unexposed samples and there is also a secure wired or wireless connection over which the device sends data, such as routes searched, samples exposed, failure to reach or complete routes, battery status, part malfunction, etc, and receives any updated commands from a remote, secure management system, such as new or modified search routes or modified time periods for route searches and other updates, etc. Failing any new commands, the VDrone or land-based robot can be programmed to run autonomously and gather and deposit samples for long periods of time.

### Special Recharge Station and Filter or Swab Deposit Method and Mechanism

Each VDrone or robot has a specially designed, air tight, secure compartment where it carries its payload of either filters or swabs or both. A VDrone or robot can carry as little as one or a plurality of filters and or swabs. On the outbound trip from the station the VDrone or robot transports a new, untouched and unexposed filter or swab on its inspection search route(s). The mobile device returns to its station carrying a filter or swab or both. The VDrone or robot could carry a number of filters or swabs specified to be air samples or surface samples from specific locations and times on its search route. At the recharge station, exposed samples are ejected into the secure container. In one example embodiment, the edge of the filter holder is stamped/ink sprayed with date, time, location data. This could also be in the form of a barcode for machine reading or a combination of human and machine readable code.

Exposed filters and swabs can be processed on site at the sampling location, or stored in an environmentally controlled filter compartment, which compartments could be stacked one on top of another, for pickup and delivery to a centrally located PCR and CRISPR based processing site.

The filter or swab compartment must be constructed so that filters with air samples or swab samples from different spaces or different time intervals do not cross-contaminate each other with an infectious targeted microbe(s). The purpose of the invention is to inspect specified spaces, areas and/or objects at specified times, gather samples from those spaces, areas and or objects, return samples for processing and report results to the proper authorities in a secure and timely manner.

### Clean or Disinfect Areas and Spaces

A similar drone or robot can force air over an internal UV source thus destroying the microbe as it passes through the irradiation chamber. In another embodiment, the drone or robot can shine UV light directly onto a surface. In addition, antimicrobial sprays and cleaners could also be used to eliminate the infectious microbe.

Various additional modifications of the described embodiments of the invention specifically illustrated and described herein will be apparent to those skilled in the art, particularly in light of the teachings of this invention. It is intended that the invention cover all modifications and embodiments, which fall within the spirit and scope of the invention. Thus, while preferred embodiments of the present invention have been disclosed, it will be appreciated that it is not limited thereto but may be otherwise embodied within the scope of the following claims.

What is claimed is:

1) A method for automatic sample collection to detect for target microbes comprising:
a) providing a first environment aboard a mobile device, the first environment protecting a first bio-active surface before the first surface is exposed;
b) determining, by the mobile device, that the mobile device is at a location;
c) opening, by the mobile device, at the location, the first environment, whereby the first surface becomes exposed;
e) closing, by the mobile device, a second environment aboard the mobile device, the second environment protecting the first surface after the first surface is exposed;
e) visiting a station, by the mobile device after the first surface is exposed;
f) transferring to the station, with the mobile device, the first surface, the station having a third environment protecting the first surface before an analysis of the first surface;
whereby a sample from the location is collected at the station for analysis.

2) The method of claim 1 wherein the mobile device is one of an aerial drone and a land-based robot.

3) The method of claim 1 wherein the first environment further controls, while closed, at least one of temperature, humidity, and light.

4) The method of claim 1 wherein the location is predetermined.

5) The method of claim 1 wherein the location is along a predetermined route.

6) The method of claim 1 wherein the opening is further at a time.

7) The method of claim 6 wherein the time is predetermined.

8) The method of claim 1 wherein the second environment is the first environment.

9) The method of claim 1 wherein at least one of the first and second environments is airtight while closed.

10) The method of claim 1 wherein the opening exposes the first surface to a flow of air.

11) The method of claim 10 wherein said flow of air is from a heating, ventilation, and air conditioning (HVAC) system.

12) The method of claim 10 wherein the mobile device is an aerial drone and said flow of air is from a rotor of the drone.

13) The method of claim 10 wherein said flow of air is measured.

14) The method of claim 10 wherein said flow of air is controlled.

15) The method of claim 1 further comprising:
g) communicating, by the station, to a server, a message that the first surface can be picked up for processing.

16) The method of claim 15 further comprising:
h) determining with an analyzer of the station whether an analysis of the first surface detects a predetermined microbe; and,
i) assigning an alert to the message if the microbe is detected, but not otherwise.

17) The method of claim 1 wherein a swab comprises the first surface and the first surface becoming exposed includes direct contact of the swab with a second surface of an object at the location.

18) The method of claim 1 further comprising:
g) transferring from the station, with the mobile device, a second bio-active surface to the mobile device, the second surface protected by a fourth environment before the second surface is exposed;
whereby the mobile device is restocked for a subsequent sampling.

19) The method of claim 18 wherein the fourth environment is the first environment.

20) The method of claim 18 wherein the transferring from the station further includes transferring the fourth environment

21) The method of claim 1 wherein the third environment is the second environment.

22) The method of claim 1 wherein the bio-active surface comprises a coating having a biosimilar enzyme matching a human body entry vector of a target microbe.

23) The method of claim 22 wherein the enzyme is supported by a cell membrane of a biosimilar human cell culture.

24) The method of claim 1 wherein the bio-active surface comprises a coating having a gene known to bind with a target microbe.
